SEQUENCE LISTING (43 sequences)

SEQ ID NO: 1 - protein, 347 aa, Klebsiella pneumonia
MSVMFDPETAIYPFPAKPQPLTVDEKQFYREKIKRLLRERDAVMVAHYYTDPEIQQLAEETGGCIADSLEMARFGARHSASTLLVAGVRFMGETAKILSPEKTILMPTLNAECSLDLGCPIEEFNAFCDAHPDRTVVVYANTSAAVKARADWVVTSSIAVELIDHLDSLGQKILWAPDRHLGRYVQRQTGADVLCWQGACIVHDEFKTQALMRMKALHPEAAVLVHPESPQAIVEMADAVGSTSQLIAAAKSLPQRQLIVATDRGIFYKMQQAVPEKTLLEAPTAGEGATCRSCAHCPWMAMNGLKAIAEGLEQGGAEHEIHVDEALRTGALIPLNRMLDFAATLRG

SEQ ID NO: 2 - DNA, 1044 bp, Klebsiella pneumonia
atgagcgtaatgtttgatcctgaaacggcgatttatcctttccctgctaaaccgcagccgctgaccgtcgacgaaaagcagttttaccgcgaaaaaatcaagcgcctgctgcgcgagcgcgatgccgtgatggtggcgcattactacaccgatcctgaaattcaacagctggcggaagagaccggcggctgtatcgccgactcgctggagatggcgcgctttggcgcccgccattcggcctccacgctgctggtcgccggggtgcgttttatgggggaaaccgccaaaattctcagcccggaaaagaccattttgatgccgaccctgaacgccgagtgttcattggatctgggctgtccgattgaggaattcaacgccttttgcgacgcccatcctgaccgcaccgtcgtggtctatgccaatacgtccgccgcggtgaaggcccgcgccgactgggtggtgacctccagcatcgccgtggaactcattgaccatctggatagtcttggtcaaaagatcctctgggcgccggaccgccaccttgggcgttacgttcagcgtcagaccggcgcagacgtgctgtgctggcagggggcgtgcatcgtgcacgacgagtttaaaacccaggcgttgatgcggatgaaggctctgcatcccgaagccgccgtgctggtccatcccgagtcgccgcaggcgatcgttgagatggccgatgccgtaggctccaccagccagctgattgcggcggcgaaaagcctgccccagcgccagctgatcgtggccaccgatcgcggtattttctataaaatgcagcaggcggtgccggagaaaacgctgctggaagcgcccaccgccggcgaaggggcgacctgccgcagctgcgcgcattgtccgtggatggcgatgaatggcctgaaagccattgccgaggggctcgagcagggcggcgctgaacatgaaatccatgtcgacgaagcgctgcgaaccggcgcattaattccccttaaccggatgctggattttgcggctacactacggggataa

SEQ ID NO: 3 - protein, 297 aa, Escherichia coli
MPPRRYNPDTRRDELLERINLDIPGAVAQALREDLGGTVDANNDITAKLLPENSRSHATVITRENGVFCGKRWVEEVFIQLAGDDVTIIWHVDDGDVINANQSLFELEGPSRVLLTGERTALNFVQTLSGVASKVRHYVELLEGTNTQLLDTRKTLPGLRSALKYAVLCGGGANHRLGLSDAFLIKENHIIASGSVRQAVEKASWLHPDAPVEVEVENLEELDEALKAGADIIMLDNFETEQMREAVKRTNGKALLEVSGNVTDKTLREFAETGVDFISVGALTKHVQALDLSMRFR

SEQ ID NO: 4 - DNA, 894 bp, Escherichia coli
atgccgcctcgccgctataaccctgacacccgacgtgacgagctgctggaacgcattaatctcgatatccccggcgcggtggcccaggcgctgcgggaagatttaggcggaacagtcgatgccaacaatgatattacggcaaaacttttaccggaaaattctcgctctcatgccacggtgatcacccgcgagaatggcgtcttttgcggcaaacgctgggttgaagaggtgtttattcaactggcaggcgacgatgtcaccataatctggcatgtggatgacggcgatgtcatcaatgccaatcaatccttgttcgaacttgaaggcccatcccgcgtgctgttaacgggcgaacgcactgcgcttaattttgtgcaaaccctttcaggagttgccagtaaggtacgccactatgtcgaattgctggaaggcaccaacacgcagttgttggatacgcgcaaaaccttacccggcctgcgttcagctctgaaatacgcggtactttgcggcggcggagcgaatcaccgtctggggctttctgatgccttcctgatcaaagaaaaccatattattgcctccggctcagtgcgccaggcggtcgaaaaagcgtcctggctgcacccggatgcgccagtagaagtcgaagtagagaatctggaagaacttgatgaagccctgaaagcaggagccgatatcatcatgctggataacttcgaaacagaacagatgcgcgaagccgtcaaacgcaccaacggcaaggcgctactggaagtgtctggcaacgtcactgacaaaacactgcgtgaatttgccgaaacgggcgtggactttatctccgtcggtgcgctaactaaacacgtacaagcactcgacctttcaatgcgttttcgctaa

SEQ ID NO: 5 - DNA, 42 bp, Artificial Sequence
  Primer
cattatacgaacggtacccccagttgaataaacacctcttca

SEQ ID NO: 6 - DNA, 18 bp, Artificial Sequence
  Primer
tggcggcaggctaatatt

SEQ ID NO: 7 - DNA, 41 bp, Artificial Sequence
  Primer
gttcttccagattctctacttttcgagctcggtacctaccg

SEQ ID NO: 8 - DNA, 42 bp, Artificial Sequence
  Primer
tgaagaggtgtttattcaactgggggtaccgttcgtataatg

SEQ ID NO: 9 - DNA, 21 bp, Artificial Sequence
  Primer
ataaccaccatcagttcgata

SEQ ID NO: 10 - DNA, 41 bp, Artificial Sequence
  Primer
cggtaggtaccgagctcgaaaagtagagaatctggaagaac

SEQ ID NO: 11 - protein, 347 aa, Escherichia coli
MSVMFDPDTAIYPFPPKPTPLSIDEKAYYREKIKRLLKERNAVMVAHYYTDPEIQQLAEETGGCISDSLEMARFGAKHPASTLLVAGVRFMGETAKILSPEKTILMPTLQAECSLDLGCPVEEFNAFCDAHPDRTVVVYANTSAAVKARADWVVTSSIAVELIDHLDSLGEKIIWAPDKHLGRYVQKQTGGDILCWQGACIVHDEFKTQALTRLQEEYPDAAILVHPESPQAIVDMADAVGSTSQLIAAAKTLPHQRLIVATDRGIFYKMQQAVPDKELLEAPTAGEGATCRSCAHCPWMAMNGLQAIAEALEQEGSNHEVHVDERLRERALVPLNRMLDFAATLRG

SEQ ID NO: 12 - DNA, 1044 bp, Escherichia coli
atgagcgtaatgtttgatccagacacggcgatttatcctttccccccgaagccgacgccgttaagcattgatgaaaaagcgtattaccgcgagaagataaaacgtctgctaaaagaacgtaatgcggtgatggttgcccactactataccgatcccgaaattcaacaactggcagaagaaaccggtggctgtatttctgattctctggaaatggcgcgcttcggtgcaaagcatcccgcttctactttgttagtcgctggggtgagatttatgggagaaaccgccaaaattctcagtccggaaaaaacaattctgatgccgacacttcaggctgaatgttcactggatctcggctgccctgttgaagaatttaacgcattttgcgatgcccatcccgatcgtactgtcgtcgtctacgccaacacttctgctgcggtaaaagcgcgcgcagattgggtggtaacttcaagcattgccgtcgaacttattgatcatcttgatagtttgggtgaaaaaatcatctgggcacccgacaaacatctggggcgttacgtgcaaaaacagacgggtggagacattctatgctggcagggtgcctgtattgtgcatgatgaatttaagactcaggcgttaacccgcttgcaagaagaatacccggatgctgccatactggtgcatccagaatcaccacaagctattgtcgatatggcggatgcggtcggttccaccagtcaactgatcgctgctgcgaaaacattgccacatcagaggcttattgtggcaaccgatcggggtattttctacaaaatgcagcaggcggtgccagataaagagttactggaagcaccaaccgcaggtgagggtgcaacctgccgcagctgcgcgcattgtccgtggatggccatgaatggccttcaggccatcgcagaggcattagaacaggaaggaagcaatcacgaggttcatgttgatgaaaggctgcgagagagggcgctggtgccgctcaatcgtatgctggattttgcggctacactacgtggataa

SEQ ID NO: 13 - DNA, 39 bp, Artificial Sequence
  Primer
cattatacgaacggtacccccccggatgctgccatactg

SEQ ID NO: 14 - DNA, 20 bp, Artificial Sequence
  Primer
ccatgagagatcataaccgc

SEQ ID NO: 15 - DNA, 40 bp, Artificial Sequence
  Primer
tgacttttccccaccattcgttcgagctcggtacctaccg

SEQ ID NO: 16 - DNA, 39 bp, Artificial Sequence
  Primer
cagtatggcagcatccgggggggtaccgttcgtataatg

SEQ ID NO: 17 - DNA, 20 bp, Artificial Sequence
  Primer
gggtcgttagctcagttggt

SEQ ID NO: 18 - DNA, 40 bp, Artificial Sequence
  Primer
cggtaggtaccgagctcgaacgaatggtggggaaaagtca

SEQ ID NO: 19 - protein, 540 aa, Escherichia coli
MNTLPEHSCDVLIIGSGAAGLSLALRLADQHQVIVLSKGPVTEGSTFYAQGGIAAVFDETDSIDSHVEDTLIAGAGICDRHAVEFVASNARSCVQWLIDQGVLFDTHIQPNGEESYHLTREGGHSHRRILHAADATGREVETTLVSKALNHPNIRVLERSNAVDLIVSDKIGLPGTRRVVGAWVWNRNKETVETCHAKAVVLATGGASKVYQYTTNPDISSGDGIAMAWRAGCRVANLEFNQFHPTALYHPQARNFLLTEALRGEGAYLKRPDGTRFMPDFDERGELAPRDIVARAIDHEMKRLGADCMFLDISHKPADFIRQHFPMIYEKLLGLGIDLTQEPVPIVPAAHYTCGGVMVDDHGRTDVEGLYAIGEVSYTGLHGANRMASNSLLECLVYGWSAAEDITRRMPYAHDISTLPPWDESRVENPDERVVIQHNWHELRLFMWDYVGIVRTTKRLERALRRITMLQQEIDEYYAHFRVSNNLLELRNLVQVAELIVRCAMMRKESRGLHFTLDYPELLTHSGPSILSPGNHYINR

SEQ ID NO: 20 - DNA, 1623 bp, Escherichia coli
atgaatactctccctgaacattcatgtgacgtgttgattatcggtagcggcgcagccggactttcactggcgctacgcctggctgaccagcatcaggtcatcgttctaagtaaaggcccggtaacggaaggttcaacattttatgcccagggcggtattgccgccgtgtttgatgaaactgacagcattgactcgcatgtggaagacacattgattgccggggctggtatttgcgatcgccatgcagttgaatttgtcgccagcaatgcacgatcctgtgtgcaatggctaatcgaccagggggtgttgtttgatacccacattcaaccgaatggcgaagaaagttaccatctgacccgtgaaggtggacatagtcaccgtcgtattcttcatgccgccgacgccaccggtagagaagtagaaaccacgctggtgagcaaggcgctgaaccatccgaatattcgcgtgctggagcgcagcaacgcggttgatctgattgtttctgacaaaattggcctgccgggcacgcgacgggttgttggcgcgtgggtatggaaccgtaataaagaaacggtggaaacctgccacgcaaaagcggtggtgctggcaaccggcggtgcgtcgaaggtttatcagtacaccaccaatccggatatttcttctggcgatggcattgctatggcgtggcgcgcaggctgccgggttgccaatctcgaatttaatcagttccaccctaccgcgctatatcacccacaggcacgcaatttcctgttaacagaagcactgcgcggcgaaggcgcttatctcaagcgcccggatggtacgcgttttatgcccgattttgatgagcgcggcgaactggccccgcgcgatattgtcgcccgcgccattgaccatgaaatgaaacgcctcggcgcagattgtatgttccttgatatcagccataagcccgccgattttattcgccagcatttcccgatgatttatgaaaagctgctcgggctggggattgatctcacacaagaaccggtaccgattgtgcctgctgcacattatacctgcggtggtgtaatggttgatgatcatgggcgtacggacgtcgagggcttgtatgccattggcgaggtgagttataccggcttacacggcgctaaccgcatggcctcgaattcattgctggagtgtctggtctatggctggtcggcggcggaagatatcaccagacgtatgccttatgcccacgacatcagtacgttaccgccgtgggatgaaagccgcgttgagaaccctgacgaacgggtagtaattcagcataactggcacgagctacgtctgtttatgtgggattacgttggcattgtgcgcacaacgaagcgcctggaacgcgccctgcggcggataaccatgctccaacaagaaatagacgaatattacgcccatttccgcgtctcaaataatttgctggagctgcgtaatctggtacaggttgccgagttgattgttcgctgtgcaatgatgcgtaaagagagtcgggggttgcatttcacgctggattatccggaactgctcacccattccggtccgtcgatcctttcccccggcaatcattacataaacagataa

SEQ ID NO: 21 - DNA, 29 bp, Artificial Sequence
  Primer
aattcatatgaatactctccctgaacatt

SEQ ID NO: 22 - DNA, 32 bp, Artificial Sequence
  Primer
aattggatccctataccactacgcttgatcac

SEQ ID NO: 23 - DNA, 23 bp, Artificial Sequence
  Primer
ctgcagatcctgcacgacccacc

SEQ ID NO: 24 - DNA, 23 bp, Artificial Sequence
  Primer
tctagacttaccatctcgtttta

SEQ ID NO: 25 - DNA, 23 bp, Artificial Sequence
  Primer
tctagaatgagcgtaatgtttga

SEQ ID NO: 26 - DNA, 23 bp, Artificial Sequence
  Primer
ggatccttatccacgtagtgtag

SEQ ID NO: 27 - DNA, 1356 bp, Artificial Sequence
  pNadA-nadA
ctgcagatcctgcacgacccaccaatgtaaaaaagcgccctaaaggcgcttttttgctattcaggcatcctcaatttcactttgtaaacctgatgacatcgtcagagcttactgtgcaagcaactctatgtcggtggaattaggcgtaaaatgacgcatcctgcacattaggcgtaattcgagtgacttttccccaccattcgactatcttgtttagcatataaaacaaattacaccgataacagcgaatattacgctaatgtcggttttaacgttaagcctgtaaaacgagatggtaagtctagaatgagcgtaatgtttgatccagacacggcgatttatcctttccccccgaagccgacgccgttaagcattgatgaaaaagcgtattaccgcgagaagataaaacgtctgctaaaagaacgtaatgcggtgatggttgcccactactataccgatcccgaaattcaacaactggcagaagaaaccggtggctgtatttctgattctctggaaatggcgcgcttcggtgcaaagcatcccgcttctactttgttagtcgctggggtgagatttatgggagaaaccgccaaaattctcagtccggaaaaaacaattctgatgccgacacttcaggctgaatgttcactggatctcggctgccctgttgaagaatttaacgcattttgcgatgcccatcccgatcgtactgtcgtcgtctacgccaacacttctgctgcggtaaaagcgcgcgcagattgggtggtaacttcaagcattgccgtcgaacttattgatcatcttgatagtttgggtgaaaaaatcatctgggcacccgacaaacatctggggcgttacgtgcaaaaacagacgggtggagacattctatgctggcagggtgcctgtattgtgcatgatgaatttaagactcaggcgttaacccgcttgcaagaagaatacccggatgctgccatactggtgcatccagaatcaccacaagctattgtcgatatggcggatgcggtcggttccaccagtcaactgatcgctgctgcgaaaacattgccacatcagaggcttattgtggcaaccgatcggggtattttctacaaaatgcagcaggcggtgccagataaagagttactggaagcaccaaccgcaggtgagggtgcaacctgccgcagctgcgcgcattgtccgtggatggccatgaatggccttcaggccatcgcagaggcattagaacaggaaggaagcaatcacgaggttcatgttgatgaaaggctgcgagagagggcgctggtgccgctcaatcgtatgctggattttgcggctacactacgtggataaggatcc

SEQ ID NO: 28 - DNA, 23 bp, Artificial Sequence
  primer
tctagaatgagcgtaatgtttga

SEQ ID NO: 29 - DNA, 23 bp, Artificial Sequence
  primer
ggatccttatccccgtagtgtag

SEQ ID NO: 30 - DNA, 1356 bp, Artificial Sequence
  pNadA-nadA(KP)
ctgcagatcctgcacgacccaccaatgtaaaaaagcgccctaaaggcgcttttttgctattcaggcatcctcaatttcactttgtaaacctgatgacatcgtcagagcttactgtgcaagcaactctatgtcggtggaattaggcgtaaaatgacgcatcctgcacattaggcgtaattcgagtgacttttccccaccattcgactatcttgtttagcatataaaacaaattacaccgataacagcgaatattacgctaatgtcggttttaacgttaagcctgtaaaacgagatggtaagtctagaatgagcgtaatgtttgatcctgaaacggcgatttatcctttccctgctaaaccgcagccgctgaccgtcgacgaaaagcagttttaccgcgaaaaaatcaagcgcctgctgcgcgagcgcgatgccgtgatggtggcgcattactacaccgatcctgaaattcaacagctggcggaagagaccggcggctgtatcgccgactcgctggagatggcgcgctttggcgcccgccattcggcctccacgctgctggtcgccggggtgcgttttatgggggaaaccgccaaaattctcagcccggaaaagaccattttgatgccgaccctgaacgccgagtgttcattggatctgggctgtccgattgaggaattcaacgccttttgcgacgcccatcctgaccgcaccgtcgtggtctatgccaatacgtccgccgcggtgaaggcccgcgccgactgggtggtgacctccagcatcgccgtggaactcattgaccatctggatagtcttggtcaaaagatcctctgggcgccggaccgccaccttgggcgttacgttcagcgtcagaccggcgcagacgtgctgtgctggcagggggcgtgcatcgtgcacgacgagtttaaaacccaggcgttgatgcggatgaaggctctgcatcccgaagccgccgtgctggtccatcccgagtcgccgcaggcgatcgttgagatggccgatgccgtaggctccaccagccagctgattgcggcggcgaaaagcctgccccagcgccagctgatcgtggccaccgatcgcggtattttctataaaatgcagcaggcggtgccggagaaaacgctgctggaagcgcccaccgccggcgaaggggcgacctgccgcagctgcgcgcattgtccgtggatggcgatgaatggcctgaaagccattgccgaggggctcgagcagggcggcgctgaacatgaaatccatgtcgacgaagcgctgcgaaccggcgcattaattccccttaaccggatgctggattttgcggctacactacggggataaggatcc

SEQ ID NO: 31 - DNA, 31 bp, Artificial Sequence
  primer
aattgggcccatgagcgtaatgtttgatcca

SEQ ID NO: 32 - DNA, 29 bp, Artificial Sequence
  primer
aattgcggccgctcgtgcctaccgcttcg

SEQ ID NO: 33 - DNA, 32 bp, Artificial Sequence
  primer
ccgcggatcccaccgcgggcttattccattac

SEQ ID NO: 34 - DNA, 34 bp, Artificial Sequence
  primer
gatgggcccatcttaatctcctagattgggtttc

SEQ ID NO: 35 - DNA, 31 bp, Artificial Sequence
  primer
aattgggcccatgagcgtaatgtttgatcct

SEQ ID NO: 36 - DNA, 29 bp, Artificial Sequence
  primer
aattgcggccgctcgtccccacggcctct

SEQ ID NO: 37 - DNA, 3096 bp, Escherichia coli
ataaggcgctcgcgccgcatccggcactgttgccaaactccagtgccgcaataatgtcggatgcgatacttgcgcatcttatccgacctacacctttggtgttacttggggcgattttttaacatttccataagttacgcttatttaaagcgtcgtgaatttaatgacgtaaattcctgctatttattcgtttgctgaagcgatttcgcagcatttgacgtcaccgcttttacgtggctttataaaagacgacgaaaagcaaagcccgagcatattcgcgccaatgcgacgtgaaggatacagggctatcaaacgataagatggggtgtctggggtaatatgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaatcttcctcttctgcaaaccctcgtgcttttgcgcgagggttttctgaaatacttctgttctaacaccctcgttttcaatatatttctgtctgcattttattcaaa

SEQ ID NO: 38 - DNA, 37 bp, Artificial Sequence
  primer
aagcttctgtaggccggataaggcgctcgcgccgcat

SEQ ID NO: 39 - DNA, 27 bp, Artificial Sequence
  primer
cggatcctttgaataaaatgcagacag

SEQ ID NO: 40 - DNA, 1556 bp, Escherichia coli
gtccacctatgttgactacatcatcaaccagatcgattctgacaacaaactgggcgtaggttcagacgacaccgttgctgtgggtatcgtttaccagttctaatagcacacctctttgttaaatgccgaaaaaacaggactttggtcctgttttttttataccttccagagcaatctcacgtcttgcaaaaacagcctgcgttttcatcagtaatagttggaattttgtaaatctcccgttaccctgatagcggacttcccttctgtaaccataatggaacctcgtcatgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaagcattaaaaacaatgaagcccgctgaaaagcgggctgagactgatgacaaacgcaacattgcctgatgcgctacgctt

SEQ ID NO: 41 - DNA, 26 bp, Artificial Sequence
  primer
ggatccgtccacctatgttgactaca

SEQ ID NO: 42 - DNA, 31 bp, Artificial Sequence
  primer
ggtaccgagctcataagcgtagcgcatcagg

SEQ ID NO: 43 - DNA, 9193 bp, Artificial Sequence
  pCPA
cccgtcttactgtcgggaattcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttctgtaggccggataaggcgctcgcgccgcatccggcactgttgccaaactccagtgccgcaataatgtcggatgcgatacttgcgcatcttatccgacctacacctttggtgttacttggggcgattttttaacatttccataagttacgcttatttaaagcgtcgtgaatttaatgacgtaaattcctgctatttattcgtttgctgaagcgatttcgcagcatttgacgtcaccgcttttacgtggctttataaaagacgacgaaaagcaaagcccgagcatattcgcgccaatgcgacgtgaaggatacagggctatcaaacgataagatggggtgtctggggtaatatgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggaccctcgcgtcgaacaagcgttaatggtcactattgccgggattgcggcaggtatgcgtaataccggctaatcttcctcttctgcaaaccctcgtgcttttgcgcgagggttttctgaaatacttctgttctaacaccctcgttttcaatatatttctgtctgcattttattcaaaggatccgtccacctatgttgactacatcatcaaccagatcgattctgacaacaaactgggcgtaggttcagacgacaccgttgctgtgggtatcgtttaccagttctaatagcacacctctttgttaaatgccgaaaaaacaggactttggtcctgttttttttataccttccagagcaatctcacgtcttgcaaaaacagcctgcgttttcatcagtaatagttggaattttgtaaatctcccgttaccctgatagcggacttcccttctgtaaccataatggaacctcgtcatgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaagcattaaaaacaatgaagcccgctgaaaagcgggctgagactgatgacaaacgcaacattgcctgatgcgctacgcttatgagctcggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggctta

(19) United States
(12) Patent Application Publication
(10) Pub. No.: US 2018/0163186 A1
(43) Pub. Date: Jun. 14, 2018

(54) MICROORGANISM HAVING QUINOLINIC ACID PRODUCTION ABILITY, AND METHOD FOR PRODUCING QUINOLINIC ACID BY USING SAME

(71) Applicant: CJ Cheiljedang Corporation, Seoul (KR)

(72) Inventors: Jaemin Lee, Uijeongbu-si, Gyeonggi-do (KR); Ju Eun Kim, Seoul (KR); Jae Hee Lee, Seoul (KR); Jin Sook Chang, Suwon-si, Gyeonggi-do (KR); So Young Kim, Gwacheon-si, Gyeonggi-do (KR); Yong Uk Shin, Yongin-si, Gyeonggi-do (KR)

(73) Assignee: CJ Cheiljedang Corporation, Seoul (KR)

(21) Appl. No.: 15/546,467

(22) PCT Filed: Dec. 28, 2015
(86) PCT No.: PCT/KR2015/014344

§ 371 Date: Jul. 26, 2017

(30) Foreign Application Priority Data
Feb. 3, 2015 (KR) KR10-2015-0016971

(51) Int. Cl. C12N 9/10; C12P 17/12; C12N 9/06

(52) U.S. Cl. CPC C12N 9/1085; C12P 17/12; C12Y 104/03016; C12N 9/0022; C12Y 205/01072

(57) ABSTRACT

The present disclosure relates to a microorganism having producing ability of quinolinic acid and a method for producing quinolinic acid using the microorganism.

### TECHNICAL FIELD

The present disclosure relates to a microorganism having producing ability of quinolinic acid and a method for producing quinolinic acid using the microorganism.

### BACKGROUND ART

Quinolinic acid is known as 2,3-pyridinedicarboxylic acid and is used as a precursor of chemical compounds used in a wide variety of fields such as medicine, agricultural chemicals, and dyeing materials.

The quinolinic acid can be prepared through chemical or biological synthesis methods. Since chemical synthesis methods use non-renewable materials derived from petroleum as raw materials, it has problems that are greatly affected by environmental problems and oil prices or petroleum extraction costs.

As a representative example of biological synthesis methods, there is a method for producing quinolinic acid in which genes encoding L-aspartate oxidase (NadB) and quinolinate synthase (NadA) are cloned into plasmids each having different copy numbers, and after enhancing the expression of the two enzymes in Escherichia coli in which the activity of quinolinate phosphoribosyltransferase is removed, quinolinic acid is produced from the strain (Eur J. Biochem. 175, 221-228 (1988), DE3826041). However, the concentration of quinolinic acid in this case was 500 mg/L or less, which was a very low level.

The first cause for the production of quinolinic acid at such a low concentration is the inhibition of transcriptional stage expression regulation by NadR, which is an NAD-related transcriptional stage inhibition factor of nadB encoding L-aspartate oxidase and nadA gene encoding quinolinate synthase. The second cause is feedback inhibition of NadB protein, which is an L-aspartate oxidase, by NAD, and the third cause is determined to be that Escherichia coli used by itself has a weak biosynthetic pathway from carbon sources to L-aspartate.

### DISCLOSURE

### Technical Problem

As a method for solving the first cause of the problem, the present inventors discovered a highly active exogenous quinolinate synthase and have made extensive efforts to increase the production amount of quinolinic acid using the same. As a result, when the activity of quinolinate synthase derived from Klebsiella pneumoniae was introduced into a microorganism having producing ability of quinolinic acid, it was found that the producing ability of quinolinic acid was more excellent compared to when quinolinate synthase derived from Escherichia coli was used, thereby completing the present disclosure.

### Technical Solution

An object of the present disclosure is to provide a microorganism having quinolinic acid productivity, which is transformed to have the activity of quinolinate synthase derived from Klebsiella pneumoniae.

Another object of the present disclosure is to provide a method for producing quinolinic acid using the microorganism having the producing ability of quinolinic acid.

### Advantageous Effects

The microorganism having the producing ability of quinolinic acid of the present disclosure can be valuably used for effective production of quinolinic acid.

### BEST MODE

A specific aspect of the present disclosure is a microorganism having producing ability of quinolinic acid, which is transformed to have the activity of quinolinate synthase derived from Klebsiella pneumoniae.

As used herein, the term “quinolinate synthase” refers to an enzyme having an activity of synthesizing quinolinic acid from iminosuccinic acid. The EC number of the quinolinate synthase is 2.5.1.72, and it is also named as NadA. The activity of the quinolinate synthase is as follows.

α-iminosuccinate+dihydroxyacetone phosphate<=>quinolinic acid+phosphate+2H₂O. [Activity of quinolinate synthase]

The quinolinate synthase which is used in the present disclosure is quinolinate synthase derived from Klebsiella, and specifically, may be quinolinate synthase derived from Klebsiella pneumoniae. In the present disclosure, the quinolinate synthase derived from Klebsiella pneumoniae is also named as NadA(KP).

The sequence of the quinolinate synthase derived from Klebsiella pneumoniae can be easily obtained from databases known in the art such as the National Center for Biotechnology Information (NCBI) and DNA Data Bank of Japan (DDBJ), and examples include, but are not limited to, the sequence of a gene having the NCBI GenBank registration number of 339761016.

The quinolinate synthase derived from Klebsiella pneumoniae may be a protein having an amino acid sequence of SEQ ID NO: 1. Furthermore, as a protein having a homology of 80% or more, specifically 90% or more, more specifically 95% or more, and even more specifically 99% or more, to SEQ ID NO: 1, if it is an amino acid sequence having a biological activity substantially identical or corresponding to quinolinate synthase derived from Klebsiella pneumoniae, it is obvious that cases where some of the sequence has deletion, modification, substitution, or addition of amino acid sequences are included within the scope of the present disclosure.

In addition, a polynucleotide encoding the quinolinate synthase derived from Klebsiella pneumoniae may have a nucleotide sequence encoding the amino acid sequence of SEQ ID NO: 1. The polynucleotide may have various modifications in a coding region within a range that does not change the amino acid sequence of a protein, due to degeneracy of a codon or considering codon preference in an organism in which the protein is to be expressed. The polynucleotide sequence may have, for example, a nucleotide sequence of SEQ ID NO: 2 and may have a homology of 80% or more, specifically 90% or more, more specifically 99% or more, but is not limited thereto.

As used herein, the term “homology” refers to the percentage of identity between two polynucleotides or polypeptide moieties. The homology between sequences from one moiety to another can be determined by known techniques. For example, homology can be determined by directly aligning the sequence information between two polynucleotide molecules or two polypeptide molecules using an easily available computer program that is capable of aligning sequence information. The computer program may be BLAST (NCBI), CLC Main Workbench (CLC bio), MegAlign™ (DNASTAR, Inc.), etc. Further, homology between polynucleotides can be determined by hybridization of the polynucleotides under conditions that result in a stable double strand between homologous regions, followed by degradation by a single-strand-specific nuclease to determine the size of degraded fragments.

Specifically, the microorganism having producing ability of quinolinic acid transformed to have activity of quinolinate synthase derived from Klebsiella pneumoniae may have enhanced activity of quinolinate synthase compared to its endogenous activity.

As used herein, the term “endogenous activity” refers to an active state of a protein of interest in its native state, i.e., in its non-variable state. “Enhanced, compared to endogenous activity” refers to an increase in activity when compared to the activity of the protein in its native state, and is a concept that also includes introducing the activity into a microorganism having no activity of the protein.

Enhancement of such activity can be accomplished by a variety of methods well known in the art, and for example, a method of inserting a polynucleotide comprising a nucleotide sequence encoding the protein into a chromosome, a method of introducing a polynucleotide encoding the protein into a vector system and thereby introducing it into a microorganism, a method of introducing a promoter having enhanced expression ability upstream of a polynucleotide encoding the protein or introducing a protein in which modifications are applied to a promoter, a method of introducing a variant of a polynucleotide encoding the protein, etc. may be used. Further, when the microorganism has the activity of the protein, a method of modifying the expression regulation sequence of a gene encoding the protein or a method of introducing a modification into a gene on a chromosome encoding the protein in order to enhance the activity of the protein, etc. may be performed, but the method is not limited by the above examples. Further, methods for enhancing such activity can be equally referenced when enhancing the activity of other proteins in the present specification.

For example, as the promoter having enhanced expression ability, known promoters may be used, and for example, the cj1 promoter (Korean Registered Patent No. 0620092), lac promoter, trp promoter, trc promoter, tac promoter, lambda phage PR promoter, PL promoter, and tet promoter may be included.

As used herein, the term “vector” refers to a DNA product containing a nucleotide sequence of a polynucleotide encoding a target protein operably linked to a suitable regulatory sequence so as to be capable of expressing the target protein within an appropriate host. The regulatory sequence includes a promoter capable of initiating transcription, any operator sequence for regulating such transcription, a sequence encoding a suitable mRNA ribosome binding site, and a sequence regulating the termination of transcription and translation. A vector may be transformed into a suitable host, then may be replicated or function, independent of the host genome, and may be integrated into the genome itself.

The vector used in the present disclosure is not particularly limited as long as it is replicable in a host, and any vector known in the art can be used. Examples of conventionally used vectors include plasmids, cosmids, viruses, and bacteriophages in their native or recombinant state. For example, pWE15, M13, AMBL3, λMBL4, λIXII, λASHII, λLAPII, λt10, λt11, Charon4A, and Charon21A vector, etc. may be used as a phage vector or cosmid vector, and PBR, pUC, pBluescriptII, pGEM, pTZ, pCL, pET vector, etc. may be used as a plasmid vector. Specifically, pACYC177, pACYC184, pCL, pECCG117, pUC19, pBR322, pMW118, PCC1BAC vector, etc. can be used. However, the vector is not limited thereto.

As used herein, the term “transformation” refers to a series of operations in which a vector containing a polynucleotide encoding a target protein is introduced into a host cell so that a protein encoded by the polynucleotide can be expressed in the host cell. The polynucleotide introduced into the host cell may be in any form as long as it can be introduced into the host cell and expressed. For example, the polynucleotide may be introduced into the host cell in the form of an expression cassette which is a structure comprising all elements required to be self-expressed (a promoter operably linked to the polynucleotide, a transcription termination signal, a ribosome binding site, a translation termination signal, etc.), and the expression cassette may be in the form of an expression vector capable of self-replication. Further, the polynucleotide may also be introduced into the host cell in its own form and thereby be operably linked to the sequence necessary for expression in the host cell.

In addition, as used above, the term “operably linked” means that a promoter sequence, which initiates and mediates transcription of a polynucleotide encoding a target protein of the present disclosure, and the gene sequence are functionally linked.

Enhancement of such activity of the protein may be such that the activity or concentration of the corresponding protein is generally increased by at least 1%, 10%, 25%, 50%, 75%, 100%, 150%, 200%, 300%, 400%, or 500%, up to 1,000% or 2,000%, based on the activity or concentration in the initial microorganism strain, but is not limited thereto.

In addition, the microorganism having producing ability of quinolinic acid may further have an enhanced activity of L-aspartate oxidase as compared to its endogenous activity.

As used herein, the term “L-aspartate oxidase” refers to an enzyme having an activity of oxidizing L-aspartate to iminosuccinate. The L-aspartate oxidase has an EC number of 1.4.3.16 and can be named as NadB. The activity of L-aspartate oxidase is as follows.

L-aspartate+fumarate<=>α-iminosuccinate+succinate+H⁺

Or

L-aspartate+oxygen<=>hydrogen peroxide+α-iminosuccinate+H⁺ [Activity of L-aspartate oxidase]

In addition, the sequence of the L-aspartate oxidase can be easily obtained from the genome sequence of Escherichia coli disclosed in the reference (Mol. Syst. Biol. 2006; 2:2006.0007. Epub 2006 Feb. 21) or from databases known in the art such as NCBI and DDBJ.

As an example, the L-aspartate oxidase may not only be a protein having an amino acid sequence of SEQ ID NO: 19, but also be a protein having a homology of 80% or more, specifically 90% or more, more specifically 95% or more, and even more specifically 99% or more, but is not limited thereto. Substantially, as long as it is an amino acid sequence having a biological activity identical or corresponding to the L-aspartate oxidase, it is obvious that cases where some of the sequence has deletion, modification, substitution, or addition of amino acid sequences are included within the scope of the present disclosure.

In addition, a polynucleotide encoding the L-aspartate oxidase may have a nucleotide sequence encoding an amino acid sequence of SEQ ID NO: 19. The polynucleotide may have various modifications in a coding region within a range that does not change the amino acid sequence of the protein, due to degeneracy of a codon or considering the codon which is preferred in an organism in which the protein is to be expressed. Further, the polynucleotide sequence may have a polynucleotide sequence of SEQ ID NO: 20, and may have a nucleotide sequence with a homology of 80% or more, specifically 90% or more, and more specifically 95% or more. However, the polynucleotide sequence is not limited thereto.

In addition, the microorganism of the genus Escherichia may further have a weakened activity of quinolinate phosphoribosyltransferase compared to its endogenous activity.

The quinolinate phosphoribosyltransferase refers to an enzyme having an activity of converting quinolinic acid into nicotinic acid mononucleotide. The EC number of the quinolinate phosphoribosyltransferase is 2.4.2.19 and is also named as NadC. The activity of quinolinate phosphoribosyltransferase is expressed as follows.

5-phospho-α-D-ribose 1-diphosphate+quinolinic acid+2H⁺<=>CO₂+diphosphate+nicotinate mononucleotide [Activity of quinolinate phosphoribosyltransferase]

In addition, the sequence of the quinolinate phosphoribosyltransferase can be easily obtained from the genome sequence (GI: 89106990) of Escherichia coli disclosed in the reference (Mol. Syst. Biol. 2006; 2:2006.2007. Epub 2006 Feb. 21) or databases known in the art such as NCBI and DDBJ, and as an example, it may not only have an amino acid sequence represented by SEQ ID NO: 3, but also have a protein having a homology of 80% or more, specifically 90% or more, more specifically 95% or more, and even more specifically 99% or more, but is not limited thereto.

In addition, the polynucleotide encoding the quinolinate phosphoribosyltransferase may have a nucleotide sequence encoding an amino acid sequence of SEQ ID NO: 3. The polynucleotide may have various modifications in a coding region within a range that does not change the amino acid sequence of the protein, due to degeneracy of a codon or considering the codon which is preferred in an organism in which the protein is to be expressed. Further, the polynucleotide sequence may have, for example, a polynucleotide sequence of SEQ ID NO: 4, and may have a nucleotide sequence with a homology of 80% or more, specifically 90% or more, more specifically 95% or more, but is not limited thereto.

By weakening the activity of NadC compared to its endogenous activity, the accumulation of quinolinic acid in cell can be increased.

The weakening of the activity of the protein compared to the endogenous activity is a concept including both cases where the activity is decreased or the activity is absent, compared to the activity of the protein of the original microorganism in its native state.

Such weakening of protein activity can be achieved by applying various methods well known in the field. As examples of the method, there are a method of deleting all or part of a gene on a chromosome encoding the protein including a case where the activity of the protein is removed; a method of replacing a gene encoding the protein on a chromosome with a gene mutated to reduce the activity of the enzyme; a method of introducing modifications into the expression regulation sequence of a gene on a chromosome encoding the protein; a method of replacing the expression regulation sequence of a gene encoding the protein with a sequence with weak activity or without activity (for example, a method of replacing the promoter of the gene with a promoter which is weaker than the endogenous promoter); a method of introducing an antisense oligonucleotide (for example, antisense RNA) which binds complementarily to a transcript of a gene on the chromosome and inhibits the translation of the mRNA to a protein; a method of artificially adding a sequence complementary to an SD sequence to the front of the SD sequence of the gene encoding the protein to form a secondary structure to make it impossible for ribosomes to attach; and a reverse transcription engineering (RTE) method in which a promoter is added to reverse the 3′ end of the open reading frame (ORF) of the corresponding sequence, etc., and it can also be achieved by a combination of these, but is not particularly limited by the above examples.

Specifically, the method of deleting all or part of a gene encoding a protein can be performed by replacing the polynucleotide encoding an endogenous target protein in a chromosome with a polypeptide, in which some nucleic acid sequences have been deleted, or a marker gene, through a vector for insertion into the chromosome in the microorganism. As an example of such a method, a method of deleting a gene by homologous recombination may be used, but is not limited thereto. Further, in the above, the term “part” varies depending on the type of polynucleotides and can be appropriately determined by those skilled in the art, and specifically, it may be 1 to 300, more specifically 1 to 100, and even more specifically 1 to 50, but is not limited thereto.

In addition, a method of modifying an expression regulation sequence can be performed by inducing modifications in the expression regulation sequence by deletion, insertion, non-conservative or conservative substitution, or a combination thereof, in the nucleic acid sequence to further weaken the activity of the expression regulation sequence, or by replacing with a nucleic acid sequence having much weaker activity. The expression regulation sequence may include a promoter, an operator sequence, a sequence encoding a ribosome binding site, and a sequence regulating the termination of transcription and translation, but is not limited thereto.

Further, a method of modifying a gene sequence on a chromosome can be performed by inducing modifications in the sequence by deletion, insertion, non-conservative or conservative substitution, or a combination thereof, to further weaken the activity of the protein, or by replacing with an improved gene sequence to have weaker activity or with an improved gene sequence without any activity, but is not limited thereto.

In addition, the microorganism of the genus Escherichia may additionally have an enhanced activity of phosphoenolpyruvate carboxylase (PPC) or L-aspartate transaminase compared to the endogenous activity.

The phosphoenolpyruvate carboxylase is an enzyme that mediates the reaction to produce oxaloacetic acid from phosphoenolpyruvate and CO₂. The EC number of the phosphoenolpyruvate carboxylase is 4.1.1.31 and is also named as PPC.

Phosphoenolpyruvate+CO₂->oxaloacetic acid+phosphate [Activity of phosphoenolpyruvate carboxylase]

In addition, L-aspartate transaminase has an activity of synthesizing L-aspartate from phosphoenolpyruvate. The EC number of the L-aspartate transaminase is 2.6.1.1, and it can also be named as AspC or L-aspartate aminotransferase.

Oxaloacetic acid+glutamic acid<=>L-aspartic acid+2-ketoglutamic acid [Activity of L-aspartate transaminase]

The sequences of the genes ppc and aspC encoding the enzymes can be obtained from the genome sequence (gi: 89110074, GI: 89107778) disclosed in the reference (Mol. Syst. Biol. 2006; 2:2006.0007. Epub 2006 Feb. 21) or from databases such as NCBI and DDBJ.

The PPC and AspC mediate the synthesis of L-aspartic acid, which is a precursor of quinolinic acid, from phosphoenolpyruvate, and when their activity is enhanced, the production of L-aspartic acid, which is a precursor of quinolinic acid in the cell, can be increased, and thereby the production of quinolinic acid can be increased.

As used herein, the term “microorganism having producing ability of quinolinic acid” refers to a microorganism capable of producing quinolinic acid from carbon sources in a medium. Further, the microorganism producing quinolinic acid may be a recombinant microorganism. Specifically, the microorganism producing quinolinic acid is not particularly limited in its type, as long as it can produce quinolinic acid, and it may be a microorganism belonging to the genus Enterobacter, the genus Escherichia, the genus Erwinia, the genus Serratia, the genus Providencia, the genus Corynebacterium, and the genus Brevibacterium, and specifically, may be a microorganism belonging to the genus Escherichia, more specifically, may be Escherichia coli, but is not limited thereto.

Another specific aspect of the present disclosure is a method for producing quinolinic acid using the microorganism having producing ability of quinolinic acid, and further having the activity of quinolinate synthase derived from Klebsiella pneumoniae.

Specifically, the method may include (a) culturing the microorganism in a medium; and (b) recovering quinolinic acid from the cultured microorganism, medium, or both of step (a).

Culture of the microorganism may be performed according to a suitable medium and culture conditions known in the art. Such a culture process can be easily adjusted and used according to the microorganism selected by those skilled in the art. Methods of culturing include batch culture, continuous culture, and fed-batch culture, but are not limited thereto. Various methods of culturing microorganisms are disclosed, for example, in “Biochemical Engineering” by James M. Lee, Prentice-Hall International Editions, pp 138-176.

The medium used for the culture may be a medium suitably satisfying the requirements of a specific microorganism. Media for various microorganisms are disclosed in the reference (“Manual of Methods for General Bacteriology” by the American Society for Bacteriology, Washington D.C., USA, 1981). The media comprise various carbon sources, nitrogen sources, and trace element components.

Carbon sources that can be used in a medium for culturing the microorganism include carbohydrates such as glucose, sucrose, lactose, fructose, maltose, starch, and cellulose; lipids such as soybean oil, sunflower oil, castor oil, and coconut oil; fatty acids such as palmitic acid, stearic acid, and linoleic acid; glycerol; alcohols such as ethanol; and organic acids such as acetic acid, but are not limited thereto. The carbon source may be used alone or in combination.

Nitrogen sources that can be used in a medium for culturing the microorganism include organic nitrogen sources and elements such as peptone, yeast extract, gravy, malt extract, corn steep liquor (CSL), and soybean wheat; and inorganic nitrogen sources such as ammonium sulfate, ammonium chloride, ammonium phosphate, ammonium carbonate, and ammonium nitrate, but are not limited thereto. The nitrogen source may be used alone or in combination.

The medium for culturing the microorganism may include potassium dihydrogen phosphate, dipotassium hydrogen phosphate, and corresponding sodium-containing salts as a source of phosphorous. Further, it may include metal salts such as magnesium sulfate or iron sulfate. In addition, amino acids, vitamins, and suitable precursors, etc. may be included in the medium, but the medium is not limited thereto. The medium for culturing the microorganism or individual components may be added to a culture solution either batchwise or continuously.

In addition, during culture, compounds such as ammonium hydroxide, potassium hydroxide, ammonia, phosphoric acid, and sulfuric acid may be added to the microbial culture solution in an appropriate manner to adjust the pH of the culture solution. Further, bubble formation can be suppressed by using a defoaming agent such as fatty acid polyglycol ester during the culture. In order to maintain the aerobic condition of the culture solution, oxygen or an oxygen-containing gas (for example, air) may be injected into the culture solution. The temperature of the culture solution may conventionally be in a range of 20° C. to 45° C., specifically 25° C. to 40° C. The culture period may be continued until the desired amount of quinolinic acid is obtained, and may specifically be in a range of 10 hours to 160 hours.

The step of recovering quinolinic acid in the step (b) may be performed through various methods well known in the art and may include a purification step.

### DETAILED DESCRIPTION OF THE INVENTION

Hereinafter, the present disclosure will be described in detail with reference to the following exemplary embodiments. However, these exemplary embodiments are for explaining the present disclosure in more detail, and the scope of the present disclosure is not intended to be limited by these exemplary embodiments.

### Example 1. Preparation of Strain Producing Quinolinic Acid

### 1-1. Preparation of Strain in which Quinolinate Phosphoribosyltransferase is Removed

The following experiment was conducted in order to delete the nadC gene encoding quinolinate phosphoribosyltransferase involved in the degradation pathway of quinolinic acid.

Through performing PCR using the chromosomal DNA of E. coli K12 W3110 as a template, the nadC gene of the quinolinic acid degradation pathway was obtained. The nucleotide sequence information (NCBI registration number “GI: 89106990”, SEQ ID NO: 4) of the nadC gene was obtained from GenBank of the National Institutes of Health (NIH), and the amino acid sequence thereof is the same as SEQ ID NO: 3. Based on SEQ ID NO: 4, primers of SEQ ID NOS: 5 and 6, which amplify a downstream region of the nadC gene, primers of SEQ ID NOS: 7 and 8, which amplify upstream and downstream regions of nadC and loxpCm, and primers of SEQ ID NOS: 9 and 10, which amplify an upstream region, were synthesized.

PCR was performed using the chromosomal DNA of E. coli K12 W3110 as a template and using the oligonucleotides of SEQ ID NOS: 5 and 6, and 9 and 10 as primers, to amplify upstream and downstream regions of the nadC gene of 0.5 kb and 0.3 kb, respectively. Further, by using pLoxpCat2 vector, which is a plasmid vector containing loxpCm (Genbank Accession No. AJ401047) as a template, and by using the oligonucleotides of SEQ ID NOS: 7 and 8 as primers, PCR was performed to amplify the loxpCm gene having a homologous sequence of the nadC gene at both ends of 1.0 kb. PfuUltra™ DNA polymerase (Stratagene, USA) was used as a polymerase, and PCR was performed by repeating 30 times a cycle consisting of denaturation for 30 seconds at 96° C., annealing for 30 seconds at 53° C., and extension for 1 minute at 72° C.

Thereafter, PCR was performed using a nadC-upstream fragment, a nadC-downstream fragment, and a loxpCm fragment, which were obtained through the above PCR reaction, as templates, and the PCR condition was repeating 10 times a cycle consisting of denaturation for 60 seconds at 96° C., denaturation for 60 seconds at 50° C., and extension for 1 minute at 72° C., and then repeating the cycle 20 times after addition of the primers of SEQ ID NOS: 5 and 10. As a result, a nadC-deficient cassette containing 1.8 kb of nadC gene-upstream-loxpCm-downstream was obtained.

The prepared nadC-deficient cassette was transformed through electroporation on E. coli KI2 W3110 containing pKD46 which is a lambda red recombinase expression vector, and it was spread on a Luria-Bertani (LB) plate medium (10 g/L of tryptone, 5 g/L of yeast extract, 10 g/L of NaCl, and 1.5% agar) containing chloramphenicol which is a selective marker, and after incubating overnight at 37° C., a strain showing resistance to chloramphenicol was selected.

By using the selected strain as a direct template, and by using primers of SEQ ID NOS: 6 and 9, PCR was performed under the same condition, and the deletion of the nadC gene was confirmed by confirming that the size of the gene was 1.6 kb in a case of a wild-type strain, and the size of the gene was 1.3 kb in a case of the nadC-removed strain, on a 1.0% agarose gel. It was named as W3110-ΔnadC.

1-2. Preparation of Strain in which Quinolinate Synthase is Removed

In order to confirm the activity of quinolinate synthase originated from Klebsiella pneumoniae of the present disclosure, the following experiment was performed to delete nadA encoding quinolinate synthase of the microorganism itself.

Through performing PCR using the chromosomal DNA of E. coli K12 W3110 as a template, the nadA gene of quinolinate synthase was obtained. The nucleotide sequence information (NCBI registration number “GI: 89107601”, SEQ ID NO: 12) of the nadA gene was obtained from GenBank of the National Institutes of Health (NIH), and the amino acid sequence thereof is the same as SEQ ID NO: 11. Based on SEQ ID NO: 12, primers of SEQ ID NOS: 13 and 14, which amplify a downstream region of the nadA gene, primers of SEQ ID NOS: 15 and 16, which amplify upstream and downstream regions of nadA and loxpCm, and primers of SEQ ID NOS: 17 and 18, which amplify an upstream region, were synthesized.

PCR was performed using the chromosomal DNA of E. coli W3110 as a template and using primers of SEQ ID NOS: 13 and 14, and 17 and 18, to amplify upstream and downstream regions of the nadA gene of 0.5 kb and 0.5 kb, respectively. Further, by using pLoxpCat2 vector, which is a plasmid vector containing loxpCm (Genbank Accession No. AJ401047) as a template, and by using the oligonucleotides of SEQ ID NOS: 15 and 16 as primers, PCR was performed to amplify the loxpCm gene having a homologous sequence of the nadA gene at both ends of 1.0 kb. PfuUltra™ DNA polymerase (Stratagene, USA) was used as a polymerase, and PCR was performed by repeating 30 times a cycle consisting of denaturation for 30 seconds at 96° C., annealing for 30 seconds at 53° C., and extension for 1 minute at 72° C.

Thereafter, PCR was performed using a nadA-upstream fragment, a nadA-downstream fragment, and a loxpCm fragment, which were obtained through the above PCR reaction, as templates, and the PCR condition was repeating 10 times a cycle consisting of denaturation for 60 seconds at 96° C., denaturation for 60 seconds at 50° C., and extension for 1 minute at 72° C., and then repeating the cycle 20 times after addition of primers of SEQ ID NOS: 13 and 18. As a result, a nadA-deficient cassette containing 2.0 kb of nadA gene-upstream-loxpCm-downstream was obtained.

The prepared nadA-deficient cassette was transformed through electroporation on E. coli W3110-ΔnadC containing pKD46 which is a lambda red recombinase expression vector, and it was spread on a Luria-Bertani (LB) plate medium (10 g/L of tryptone, 5 g/L of yeast extract, 10 g/L of NaCl, and 1.5% agar) containing chloramphenicol which is a selective marker, and after incubating overnight at 37° C., a strain showing resistance to chloramphenicol was selected.

By using the selected strain as a direct template, and by using primers of SEQ ID NOS: 14 and 17, PCR was performed under the same condition, and the deletion of the nadA gene was confirmed by confirming that the size of the gene was 1.1 kb in a case of a wild-type strain, and the size of the gene was 1.3 kb in a case of the nadA-removed strain, on a 1.0% agarose gel. It was named as W3110-ΔnadCΔnadA.

1-3. Preparation of E. coli L-Aspartate Oxidase Expression Vector

The following experiment was performed in order to enhance the nadB gene encoding L-aspartate oxidase.

A wild-type nadB gene originated from E. coli was cloned to an expression vector. As a template therefor, the chromosome of E. coli K12 W3110 strain (ATCC No. 23257) was used. The gene sequence was based on the nucleotide sequence (NCBI registration number “GI: 89109380”, SEQ ID NO: 20) of the gene from GenBank of the National Institutes of Health (NIH), and the amino acid sequence is the same as SEQ ID NO: 19. Primers of SEQ ID NOS: 21 and 22 having recognition sites of restriction enzymes NdeI and BamHI, which amplify the ORF region of nadB gene were synthesized.

PCR was performed using the chromosomal DNA of the E. coli K12 W3110 as a template and using the oligonucleotides of SEQ ID NOS: 21 and 22 as primers. PfuUltra™ DNA polymerase (Stratagene, USA) was used as a polymerase, and PCR was performed by repeating 30 times a cycle consisting of denaturation for 30 seconds at 96° C., annealing for 30 seconds at 50° C., and extension for 2 minutes at 72° C. Through performing PCR, an amplified gene of about 1.9 kb containing the nadB ORF gene and the recognition sites of restriction enzymes NdeI and BamHI was obtained.

The nadB gene obtained through the PCR was recovered through agarose gel elution, followed by treatments with restriction enzymes NdeI and BamHI. Thereafter, it was ligated to a pProLar vector (CloneTech, USA) which was treated with restriction enzymes NdeI and BamHI, and L-aspartate oxidase was expressed from the nadB gene linked to a pPro promoter. The vector prepared by the above method was named as pPro-nadB vector.

1-4. Preparation of Vector in which Quinolinate Synthase is Expressed

(1) Preparation of pNadA-nadA Vector

The nadA gene encoding a wild-type quinolinate synthase originated from E. coli was cloned to an expression vector. As a template therefor, the chromosome of E. coli K12 W3110 strain (ATCC NO. 23257) was used. The nucleotide sequence information of the nadA gene of SEQ ID NO: 12 (NCBI registration number “GI: 89107601”) was obtained from GenBank of the National Institutes of Health (NIH). Further, based on this, primers of SEQ ID NOS: 25 and 26 having recognition sites of restriction enzymes XbaI and BamHI, which can amplify the ATG region of the nadA gene and ORF region containing TAA were synthesized.

PCR was performed using the chromosomal DNA of the E. coli W3110 as a template and using the oligonucleotides of SEQ ID NOS: 25 and 26 as primers. PfuUltra™ DNA polymerase (Stratagene, USA) was used as a polymerase, and PCR was performed by repeating 30 times a cycle consisting of denaturation for 30 seconds at 96° C., annealing for 30 seconds at 50° C., and extension for 2 minutes at 72° C. As a result, an amplified gene of about 1 kb containing the nadA gene and recognition sites of restriction enzymes XbaI and BamHI was obtained.

In addition, based on Mendoza-Vargas, et al., PLoS ONE, 4: e7526, a pNadA promoter was obtained through performing PCR using the chromosomal DNA of E. coli W3110 containing a pNadA promoter. In order to ligate the pNadA promoter and the nadA gene that is amplified above, primers of SEQ ID NOS: 23 and 24 having recognition sites of restriction enzymes PstI and XbaI were synthesized.

PCR was performed using the chromosomal DNA of the E. coli W3110 as a template and using the oligonucleotides of SEQ ID NOS: 23 and 24 as primers. PfuUltra™ DNA polymerase (Stratagene, USA) was used as a polymerase, and the PCR condition was repeating 30 times a cycle consisting of denaturation for 30 seconds at 96° C., annealing for 30 seconds at 50° C., and extension for 1 minute at 72° C. As a result, an amplified gene of about 0.3 kb containing a pNadA promoter and recognition sites of restriction enzymes PstI and XbaI was obtained.

The nadA gene obtained through the PCR was treated with restriction enzymes XbaI and BamHI, and amplified pNadA promoter fragments were treated with PstI and XbaI. The nadA and pNadA promoter fragments which were treated with the restriction enzymes were cloned into a pCL1920 vector through ligation to prepare a pNadA-nadA recombinant vector. This is the same as shown in SEQ ID NO: 27.

(2) Preparation of pNadA-nadA (KP) Vector

In order to prepare quinolinate synthase originated from Klebsiella pneumoniae, nadA(KP) gene encoding Klebsiella quinolinate synthase was cloned into an expression vector. For this, the chromosomal DNA of a Klebsiella strain was used as a template. For the strain, ATCC No. 25955 was purchased and used. The gene sequence used was SEQ ID NO: 2 of the nucleotide sequence of the gene of GenBank of the National Institutes of Health (NIH), and the amino acid sequence thereof was the same as SEQ ID NO: 1. For gene cloning, primers of SEQ ID NOS: 28 and 29 having recognition sites of restriction enzymes XbaI and BamHI capable of amplifying the nadA(KP) gene region were synthesized.

PCR was performed using the chromosomal DNA of Klebsiella as a template and using the oligonucleotides of SEQ ID NOS: 28 and 29 as primers. PfuUltra™ DNA polymerase (Stratagene, USA) was used as a polymerase, and PCR was performed by repeating 30 times a cycle consisting of denaturation for 30 seconds at 96° C., annealing for 30 seconds at 50° C., and extension for 2 minutes at 72° C. As a result, an amplified gene of about 1 kb containing the nadA(KP) gene and recognition sites of restriction enzymes XbaI and BamHI was obtained.

The nadA(KP) gene obtained through the PCR was treated with restriction enzymes XbaI and BamHI, and a pNadA-nadA(KP) recombinant vector was prepared by ligating nadA(KP) fragments treated with restriction enzymes to a pCL1920 vector containing a pNadA promoter. This nucleotide sequence is the same as shown in SEQ ID NO: 30.

1-5. Preparation of Plasmid for Expression of Aspartate Oxidase and Quinolinate Synthase

(1) Preparation of pPro-nadB-pCJ1-nadA Vector

In order to produce quinolinic acid, enhancement of two enzymes, that is, aspartate oxidase and quinolinate synthase, is necessary. Therefore, a plasmid was prepared in which the nadB and nadA genes encoding these two enzymes could be expressed together. First, the nadA gene encoding quinolinate synthase was obtained through performing PCR using the chromosomal DNA of E. coli W3110 as a template. The nucleotide sequence information of the nadA gene (NCBI registration number “GI: 89107601”) was used from GenBank of the National Institutes of Health (NIH), and it is the same as SEQ ID NO: 12. Based on this, primers of SEQ ID NOS: 31 and 32 having recognition sites of restriction enzymes ApaI and NotI, which can amplify the ATG region of the nadA gene and ORF region containing TAA were synthesized.

PCR was performed using the chromosomal DNA of E. coli W3110 as a template and using the oligonucleotides of SEQ ID NOS: 31 and 32 as primers. PfuUltra™ DNA polymerase (Stratagene, USA) was used as a polymerase, and PCR was performed by repeating 30 times a cycle consisting of denaturation for 30 seconds at 96° C., annealing for 30 seconds at 50° C., and extension for 2 minutes at 72° C. As a result, an amplified gene of about 1.0 kb containing the nadA gene and recognition sites of restriction enzymes ApaI and NotI was obtained.

Based on the Korean Registered Patent No. 0620092, a pCJ1 promoter was obtained through performing PCR using the plasmid DNA containing a pCJ1 promoter as a template. In order to ligate the nadA gene, which is amplified above, with a pCJ1 promoter, primers of SEQ ID NO: 33 and 34 having recognition sites of restriction enzymes BamHI and ApaI were synthesized.

PCR was performed using the chromosomal DNA of E. coli W3110 as a template and using the oligonucleotides of SEQ ID NOS: 33 and 34 as primers. PfuUltra™ DNA polymerase (Stratagene, USA) was used as a polymerase, and the PCR condition was repeating 30 times a cycle consisting of denaturation for 30 seconds at 96° C., annealing for 30 seconds at 50° C., and extension for 1 minute at 72° C. As a result, an amplified gene of about 0.3 kb containing the pCJ1 promoter and recognition sites of restriction enzymes BamHI and ApaI was obtained.

The nadA gene obtained through performing the PCR was treated with restriction enzymes ApaI and NotI, and the amplified pCJ1 promoter fragments were treated with ApaI and BamHI. The nadA and pCJ1 promoter fragments which were treated with the restriction enzymes were cloned into the pPro-nadB vector obtained in Example 1-3, which was treated with NotI and BamHI, through ligation, and finally a pPro-nadB_pCJ1-nadA recombinant vector of 5.9 kb was prepared, in which the nadB gene, whose expression is regulated by the pPro promoter as a constitutive promoter, and the nadA gene, whose expression is regulated by the pCJ1 gene promoter, were cloned.

(2) Preparation of pPro-nadB-pCJ1-nadA(KP) Vector

In order to produce quinolinic acid, enhancement of two enzymes, that is, aspartate oxidase and quinolinate synthase, is necessary. For the preparation of a plasmid in which nadB and nadA(KP) encoding these two enzymes could be expressed together, the nadA(KP) gene encoding quinolinate synthase was obtained through performing PCR using the chromosomal DNA of a Klebsiella strain as a template. The sequence used was the nucleotide sequence (NCBI registration number “GI: 339761016”) of the gene of GenBank of the National Institutes of Health (NIH), and it is the same as SEQ ID NO: 2. Based on this, primers of SEQ ID NOS: 35 and 36 having recognition sites of restriction enzymes ApaI and NotI, which can amplify ATG region of nadA(KP) gene and ORF region containing TAA were synthesized.

PCR was performed using the chromosomal DNA of the Klebsiella strain as a template and using the oligonucleotides of SEQ ID NOS: 35 and 36 as primers. PfuUltra™ DNA polymerase (Stratagene, USA) was used as a polymerase, and PCR was performed by repeating 30 times a cycle consisting of denaturation for 30 seconds at 96° C., annealing for 30 seconds at 50° C., and extension for 2 minutes at 72° C. As a result, an amplified gene of about 1.0 kb containing the nadA promoter and recognition sites of restriction enzymes ApaI and NotI was obtained.

The nadA(KP) gene obtained through the PCR was treated with restriction enzymes ApaI and NotI, and was cloned into the pPro-nadB-pCJ1-nadA vector obtained in Example 1-5(1) through ligation, and finally a pPro-nadB_pCJ1-nadA(KP) recombinant vector of 5.9 kb was prepared, in which the nadB gene, whose expression is regulated by the pPro promoter as a constitutive promoter, and the nadA(KP) gene, whose expression is regulated by the pCJ1 gene promoter, were cloned.

1-6. Preparation of Plasmid for Expressions of Phosphoenolpyruvate Carboxylase and L-Aspartate Transaminase

Through performing PCR using the chromosomal DNA of E. coli W3110 as a template, the ppc gene encoding phosphoenolpyruvate carboxylase was obtained. The nucleotide sequence of the ppc gene (NCBI registration number “GI: 89110074”) of SEQ ID NO: 37 was obtained from GenBank of the National Institutes of Health (NIH), and based on this, primers of SEQ ID NOS: 38 and 39 having recognition sites of restriction enzymes HindIII and BamHI, which can amplify a region containing the promoter of the ppc gene to the terminator, were synthesized.

PCR was performed using the chromosomal DNA of E. coli W3110 strain as a template and using the oligonucleotides of SEQ ID NOS: 38 and 39 as primers. PfuUltra™ DNA polymerase (Stratagene, USA) was used as a polymerase, and PCR was performed by repeating 30 times a cycle consisting of denaturation for 30 seconds at 96° C., annealing for 30 seconds at 50° C., and extension for 4 minutes at 72° C. As a result, an amplified gene of about 3.1 kb containing the ppc gene and recognition sites of restriction enzymes HindIII and BamHI was obtained. The ppc gene which was obtained through the PCR was treated with restriction enzymes HindIII and BamHI, and was cloned into a PCL920(AB236930) vector, which was treated with restriction enzymes HindIII and BamHI, through ligation, and finally, a pCP recombinant vector was prepared, in which the ppc gene was cloned.

In order to clone the aspC gene on the pCP recombinant vector in which the ppc gene was cloned, the aspC gene encoding L-aspartate transaminase was obtained through performing PCR using the chromosomal DNA of E. coli W3110 as a template. The nucleotide sequence of the aspC gene (NCBI registration number “GI 89107778”) of SEQ ID NO: 40 was obtained from GenBank of the National Institutes of Health (NIH), and based on this, primers of SEQ ID NOS: 41 and 42 having recognition sites of restriction enzymes BamHI and KpnI, which can amplify a region containing the promoter of the aspC gene to the terminator, were synthesized.

PCR was performed using the chromosomal DNA of E. coli W3110 as a template and using the oligonucleotides of SEQ ID NOS: 41 and 42 as primers. PfuUltra™ DNA polymerase (Stratagene, USA) was used as a polymerase, and PCR was performed by repeating 30 times a cycle consisting of denaturation for 30 seconds at 96° C., annealing for 30 seconds at 50° C., and extension for 2 minutes at 72° C. As a result, an amplified gene of about 1.5 kb containing the aspC gene and recognition sites of restriction enzymes BamHI and KpnI was obtained.

The aspC gene obtained through the PCR was treated with restriction enzymes BamHI and KpnI, and was cloned through ligation to the pCP vector which was treated with restriction enzymes BamHI and KpnI, and finally, a pCPA recombinant vector was prepared, in which the aspC gene and ppc gene were simultaneously cloned. The prepared pCPA vector has a sequence of SEQ ID NO: 43.

### Example 2. Evaluation of Productivity of Quinolinic Acid-Producing Strain

In order to evaluate the ability of the nadA(KP) gene-enhanced strain to produce quinolinic acid, pNadA-nadA and pNadA-nadA(KP) vectors were introduced into the W3110-ΔnadC strain, and W3110ΔnadC/pNadA-nadA and W3110ΔnadC/pNadA-nadA(KP) strains were named as CV01-0812 and CV01-0813, respectively. In addition, for the enhancement of nadB, a pPro-nadB vector was introduced into CV01-0812 and CV01-0813, and they were named as CV01-0814 and CV01-0815, respectively.

The introduction method of vectors was transformation using the CaCl₂ method, and in an incubator at 37° C., CV01-0812 and CV01-0813 were spread on an LB-Sp (10 g/L of yeast extract, 5 g/L of NaCl, 10 g/L of Tryptone, 1.5% agar, 50 μg/L of spectinomycin) plate medium and cultured overnight, and CV01-0814 and CV01-0815 were spread on an LB-sp, Km (10 g/L of yeast extract, 5 g/L of NaCl, 10 g/L of Tryptone, 1.5% agar, 50 μg/L of kanamycin, 50 μg/L of spectinomycin) plate medium and cultured overnight. Thereafter, an obtained single colony having antibiotic resistance was inoculated by a platinum loop into 25 mL of a quinolinic acid titer medium and cultured for 24 hours to 72 hours at 250 rpm at 33° C. Table 1 below represents the composition of the medium for producing quinolinic acid.

TABLE 1
Composition of quinolinic acid titer medium

| Composition | Concentration (per liter) | |
|---|---|---|
| Glucose | 70 | g |
| Ammonium sulfate | 17 | g |
| KH₂PO₄ | l.0 | g |
| MgSO₄•7 H₂O | 0.5 | g |
| FeSO₄ •7 H₂O | 5 | mg |
| MnSO₄•8 H₂O | 5 | mg |
| ZnSO₄ | 5 | mg |
| Calcium carbonate | 30 | g |
| Yeast extract | 2 | g |
| Methionine | 0.15 | g |

Quinolinic acid in the culture solution was analyzed by HPLC, and the results are shown in Table 2 below. As can be confirmed in Table 2, the necessity of simultaneous enhancement of nadB and nadA in the production of quinolinic acid was confirmed, and it was confirmed that the Klebsiella-based nadA(KP)-enhanced strain showed approximately a 10% increase in the production of quinolinic acid compared to the wild-type nadA-enhanced strain.

TABLE 2

| Strain | Quinolinic acid (g/L) |
|---|---|
| CV01-0812 | 0.1 |
| CV01-0813 | 0.1 |
| CV01-0814 | 5.6 |
| CV01-0815 | 6.1 |

To evaluate the activity of single-species quinolinate synthase, pNadA-nadA and pNadA-nadA(KP) vectors were transformed into W3110-ΔnadCΔnadA strains containing a pPro-nadB vector, respectively, using the CaCl₂ method, and W3110ΔnadCΔnadA/pNadA-nadA, pPro-nadB and W3110ΔnadCΔnadA/pNadA-nadA(KP), pPro-nadB strains were named as CV01-0816 and CV01-0817, respectively. Among these, the CV01-0817 strain was deposited under the Budapest Treaty on Nov. 27, 2014, with the Korean Culture Center of Microorganisms (KCCM) and was granted an accession number of KCCM 11612P.

Using the above two strains, evaluation of a quinolinic acid titer medium was conducted as follows.

The transformed CV01-0816 and CV01-0817 strains were spread on an LB-Km, Sp (10 g/L of yeast extract, 5 g/L of NaCl, 10 g/L of Tryptone, 1.5% agar, 50 μg/L of kanamycin, 50 μg/L of spectinomycin) plate medium and cultured overnight in an incubator at 37° C. Thereafter, an obtained single colony having kanamycin and spectinomycin resistances was inoculated by a platinum loop into 25 mL of a quinolinic acid titer medium and cultured for 24 hours to 72 hours at 250 rpm at 33° C.

Quinolinic acid in the culture solutions was analyzed by HPLC, and the results are shown in Table 3. As can be confirmed in Table 3, when Klebsiella-based nadA(KP) was enhanced, the production of quinolinic acid increased by more than 9% compared to when

TABLE 3

| Strain | Quinolinic acid (g/L) |
|---|---|
| CV01-0816 | 6.1 |
| CV01-0817 | 6.5 |

Additionally, in order to evaluate the producing ability of quinolinic acid when enhancing the biosynthetic pathway, a pCPA vector was introduced into W3110-ΔnadC and W3110-ΔnadCΔnadA strains, and further, pPro-nadB-pCJ1-nadA and pPro-nadB-pCJ1-nadA(KP) vectors, in which nadB and nadA were simultaneously enhanced, were introduced, respectively. W3110-ΔnadC/pCPA, pPro-nadB-pCJ1-nadA, W3110-ΔnadC/pCPA, pPro-nadB-pCJ1-nadA(KP), W3110-ΔnadCΔnadA/pCPA, pPro-nadB-pCJ1-nadA, and W3110-ΔnadCΔnadA/pCPA, and pPro-nadB-pCJ1-nadA(KP) strains were named as CV01-0818, CV01-0819, CV01-0820, and CV01-0821, respectively.

The introduction method was transformation using the CaCl₂ method, and these strains were spread on an LB-Km, Sp (10 g/L of yeast extract, 5 g/L of NaCl, 10 g/L of tryptone, 50 μg/L of kanamycin, 50 μg/L of spectinomycin) plate medium and cultured overnight in an incubator at 37° C. Thereafter, an obtained single colony having kanamycin and spectinomycin resistances was inoculated by a platinum loop into 25 mL of a quinolinic acid titer medium and cultured for 24 hours to 72 hours at 250 rpm at 33° C.

Quinolinic acid in the culture solutions was analyzed by HPLC, and the results are shown in Table 4 below. As can be seen in Table 4 below, it was confirmed that when Klebsiella-based nadA(KP) was enhanced, the production of quinolinic acid was increased by more than 10% compared to when nadA was enhanced.

TABLE 4

| Strain | Quinolinic acid (g/L) |
|---|---|
| CV01-0818 | 7.1 |
| CV01-0819 | 7.8 |
| CV01-0820 | 7.3 |
| CV01-0821 | 8.1 |

From the foregoing, a skilled person in the art to which the present disclosure pertains will be able to understand that the present disclosure may be embodied in other specific forms without modifying the technical concepts or essential characteristics of the present disclosure. In this regard, the exemplary embodiments disclosed herein are only for illustrative purposes and should not be construed as limiting the scope of the present disclosure. On the contrary, the present disclosure is intended to cover not only the exemplary embodiments but also various alternatives, modifications, equivalents, and other embodiments that may be included within the spirit and scope of the present disclosure as defined by the appended claims.

1. A microorganism of the genus Escherichia having producing ability of quinolinic acid, which is transformed to have an activity of quinolinate synthase derived from Klebsiella pneumoniae.

2. The microorganism of the genus Escherichia according to claim 1, wherein the quinolinate synthase derived from Klebsiella pneumoniae has an amino acid sequence of SEQ ID NO: 1.

3. The microorganism of the genus Escherichia according to claim 1, wherein a polynucleotide encoding the quinolinate synthase derived from Klebsiella pneumonia has a nucleotide sequence of SEQ ID NO: 2.

4. The microorganism of the genus Escherichia according to claim 1, wherein the microorganism of the genus Escherichia further has an enhanced activity of L-aspartate oxidase compared to its endogenous activity.

5. The microorganism of the genus Escherichia according to claim 1, wherein the microorganism of the genus Escherichia further has a weakened activity of quinolinate phosphoribosyltransferase compared to its endogenous activity.

6. The microorganism of the genus Escherichia according to claim 1, wherein the microorganism of the genus Escherichia is Escherichia coli.

7. A method for producing quinolinic acid, comprising:
(a) culturing the microorganism of the genus Escherichia of claim 1 in a medium; and
(b) recovering quinolinic acid from the cultured microorganism, the medium, or both of step (a).

8. The microorganism of the genus Escherichia according to claim 4, wherein the microorganism of the genus Escherichia further has a weakened activity of quinolinate phosphoribosyltransferase compared to its endogenous activity.

9. The method according to claim 7, wherein the quinolinate synthase derived from Klebsiella pneumoniae has an amino acid sequence of SEQ ID NO: 1.

10. The method according to claim 7, wherein a polynucleotide encoding the quinolinate synthase derived from Klebsiella pneumonia has a nucleotide sequence of SEQ ID NO: 2.

11. The method according to claim 7, wherein the microorganism of the genus Escherichia further has an enhanced activity of L-aspartate oxidase compared to its endogenous activity.

12. The method according to claim 7, wherein the microorganism of the genus Escherichia further has a weakened activity of quinolinate phosphoribosyltransferase compared to its endogenous activity.

13. The method according to claim 11, wherein the microorganism of the genus Escherichia further has a weakened activity of quinolinate phosphoribosyltransferase compared to its endogenous activity.

14. The method according to claim 7, wherein the microorganism of the genus Escherichia is Escherichia coli.
